(19) United States
(12) Patent Application Publication
(10) Pub. No.: US 2021/0213230 A1
(43) Pub. Date: Jul. 15, 2021

(54) CPAP/BIPAP MASK STRAP FACE PROTECTOR

(71) Applicant: Gordon Burry, Portugal Cove (CA)

(72) Inventor: Gordon Burry, Portugal Cove (CA)

(21) Appl. No.: 16/743,975

(22) Filed: Jan. 15, 2020

(51) Int. Cl. A61M 16/06

(52) U.S. Cl. CPC A61M 16/0683; A41D 13/11

(57) ABSTRACT

An illustrated side view of an exemplary protective covering for protecting the face of a person undergoing CPAP/BIPAP therapy is presented. The protective covering is useful for providing a soft, comfortable covering for straps of a CPAP/BIPAP mask that contacts a face of the person. The protective covering further is useful for providing a covering that can be useful for aiding in keeping the CPAP/BIPAP mask in a desired position and in full contact with the face of the person thus preventing gaps between the face and the mask that can compromise CPAP/BIPAP therapy. A CPAP/BIPAP therapy device is useful for forming a positive airway pressure ventilator, which applies mild air pressure on a continuous basis to keep the airways continuously open in people who are not able to breathe spontaneously on their own.

### FIELD OF THE INVENTION

This invention relates to CPAP/BIPAP masks. More particularly, it relates to a cover for lower straps of a CPAP/BIPAP mask.

### BACKGROUND

Continuous positive airway pressure (CPAP/BIPAP) is a form of positive airway pressure ventilator, which applies mild air pressure on a continuous basis to keep the airways continuously open in people who are not able to breathe spontaneously on their own. It is an alternative to positive end-expiratory pressure (PEEP). Both modalities stent the lungs' alveoli open and thus recruit more of the lung's surface area for ventilation, but, while PEEP refers to devices that impose positive pressure only at the end of the exhalation, CPAP/BIPAP devices apply continuous positive airway pressure throughout the breathing cycle. Thus, the ventilator itself does not cycle during CPAP/BIPAP, no additional pressure above the level of CPAP/BIPAP is provided, and patients must initiate all of their breaths.

CPAP/BIPAP typically is used for people who have breathing problems, such as sleep apnea. CPAP/BIPAP also may be used to treat preterm infants whose lungs have not yet fully developed. For example, physicians may use CPAP/BIPAP in infants with respiratory distress syndrome. It is associated with a decrease in the incidence of bronchopulmonary dysplasia. In some preterm infants whose lungs have not fully developed, CPAP/BIPAP improves survival and decreases the need for steroid treatment for their lungs. In resource-limited settings where CPAP/BIPAP improves respiratory rate and survival in children with primary pulmonary disease, researchers have found that nurses can initiate and manage care with once or twice daily physician rounds.

CPAP/BIPAP therapy utilizes machines specifically designed to deliver a constant flow of pressure. Some CPAP/BIPAP machines have other features as well, such as heated humidifiers. CPAP/BIPAP is the most effective treatment for obstructive sleep apnea, in which the mild pressure from the CPAP/BIPAP prevents the airway from collapsing or becoming blocked.

Although delivery of CPAP/BIPAP through a nasal mask is the most common modality of treatment, other systems exist for interfacing with adults and children. Nasal CPAP/BIPAP is frequently used in infants, though its use is controversial. Studies have shown nasal CPAP/BIPAP reduces ventilator time but an increased occurrence of pneumothorax was also prevalent. Oral masks and naso-oral masks are often used when nasal congestion or obstruction is an issue.

CPAP/BIPAP machines are coupled to a person by a tube that provides the therapy to a mask that fits over the person's nose and mouth. It is critical that the mask fit tightly to provide relief for the person. The CPAP/BIPAP therapy is generally provided while the person is sleeping.

The mask is coupled to the person by the mask. The mask has a strap which fits around the head and the lower part of the face of the person to secure the CPAP/BIPAP mask to the person. The CPAP/BIPAP mask can be moved while the person sleeps by normal movement of the person. The mask then can be loosened from the face of the person and the CPAP/BIPAP therapy is not effective or less effective than desired. The straps of the mask also create markings on the face of the person where the straps contact the face, these markings are not desirable and can cause issues for the person.

In light of the foregoing, there is a need for a device to provide a protective covering for the face of the person utilizing CPAP/BIPAP therapy while also helping to secure the CPAP/BIPAP mask to the face of the person.

### BRIEF DESCRIPTION OF THE DRAWINGS

FIG. 1 is an illustrated side view of an exemplary protective covering.

FIG. 2 is an illustrated view of a CPAP/BIPAP therapy device utilizing the exemplary protective covering shown in FIG. 1.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

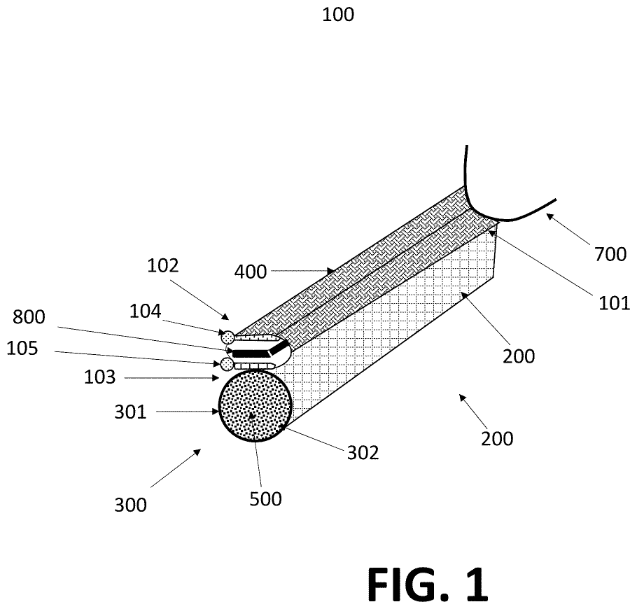

Referring to FIG. 1, an illustrated side view of an exemplary protective covering 100 for protecting the face of a person undergoing CPAP/BIPAP therapy is presented. The protective covering 100 is useful for providing a soft, comfortable covering for straps of a CPAP/BIPAP mask that contacts a face of the person. The protective covering 100 further is useful for providing a covering that can be useful for aiding in keeping the CPAP/BIPAP mask in a desired position and in full contact with the face of the person thus preventing gaps between the face and the mask that can compromise CPAP/BIPAP therapy.

The protective covering 100 has a body 200, an interior 300, a strap coupling 400 and a filling 500. The body 200 of the protective mask 100 preferably is made of a flannel material, however other materials are hereby contemplated, including, but not limited to, cotton, silk, polyester, fleece, etc. The protective covering 100 preferably has a length between four and five (4-5) inches, however other lengths are hereby contemplated, including, but not limited to, three (3) inches, six (6) inches, etc. The protective covering 100 is preferably one (1) inch in height, however other heights are hereby contemplated, including, but not limited to, one-half (0.5) inch, two (2) inches, etc. The protective covering 100 is preferably one (1) inch in width, however other widths are hereby contemplated, including, but not limited to, one-half (0.5) inch, two (2) inches, etc.

The interior 300 of the protective covering 100 has a outer edge 301 and inner edge 302. The outer edge 301 is preferably one (1) inch in diameter, however other diameters are hereby contemplated, including, but not limited to, one-half (0.5) inch, two (2) inches, etc. The inner edge 302 is preferably three-fourth (0.75) inch in diameter, however other diameters are hereby contemplated, including, but not limited to, one-half (0.5) inch, one (1) inches, etc. The outer edge 301 must have a larger diameter than the inner edge 302.

The filling 500 of the protective covering 100 is preferably made of a polyester material, however other materials are hereby contemplated, including, but not limited to, foam, down, cotton, etc. The filling 500 is configured to be in the interior 300 of the protective covering 100. To ensure that the filling 500 remains in the interior 300 of the protective covering 100 where a coupling 101 is employed to enclose the filling 500. The coupling 101 of the protective covering 100 is preferably a thread, however other couplings are hereby contemplated, including, but not limited to, adhesive, snaps, clips, etc.

The strap coupling 400 removably couples the protective 100 to the strap 800 of the CPAP/BIPAP mask 700. The strap coupling 400 of the protective covering 100 is preferably a hook and loop Velcro® coupling, however other types of couplings are hereby contemplated, including, but not limited to, snaps, clips, etc.

At a top 102 of the protective cover 100 is a first hook 103 and at a bottom 103 of the protective cover 100 is a second hook 104. When the protective covering 100 is coupled to the strap 700 of the CPAP/BIPAP mask 800, the first hook 103 is coupled to the body 200 of the protective cover 100 and the second hook 104 is coupled to the body 200 of the protective cover 100.

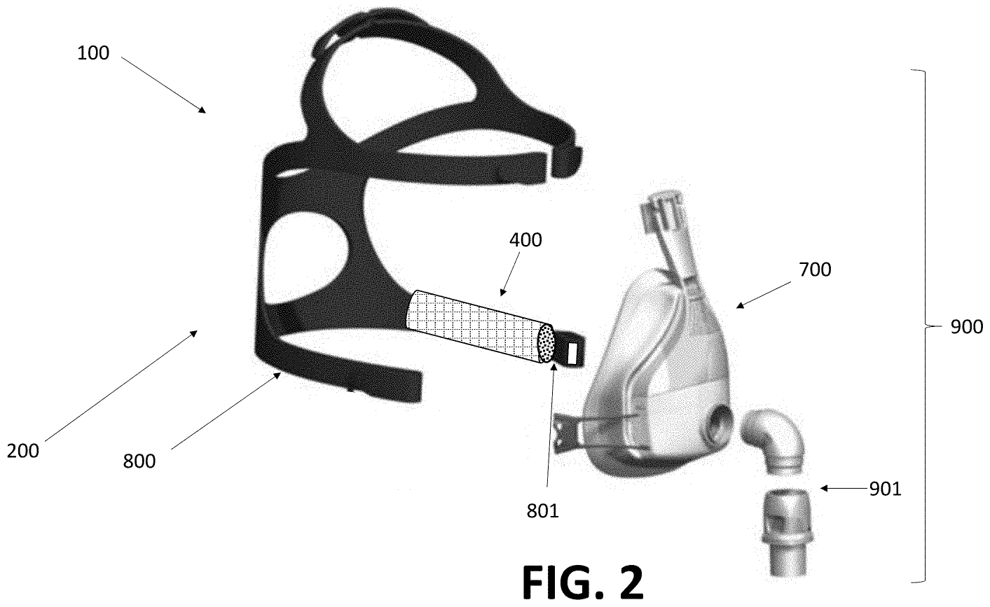

Moving now to FIG. 2, an illustrated view of a CPAP/BIPAP therapy device 900 utilizing the exemplary protective covering 100 shown in FIG. 1 is presented.

The CPAP/BIPAP therapy device 900 is useful for forming a positive airway pressure ventilator, which applies mild air pressure on a continuous basis to keep the airways continuously open in people who are not able to breathe spontaneously on their own.

The CPAP/BIPAP therapy device 900 has a tubing 901, the mask 700 and a strap 800. The strap 800 is fitted around a head of a person who is utilizing the CPAP/BIPAP therapy device 900. The strap 800 is coupled to the mask 700 such that the mask 700 is secured to a face of the person, preferably where there are no gaps between the mask 700 and the face of the person thus the CPAP/BIPAP therapy device 900 affects are not compromised.

The protective covering 100 is coupled to an inside portion 801 of the strap 700 significantly over the inside portion 801 configured to fit around the face of the person. The strap coupling 400 is preferably removably coupled to the inside portion 801 of the strap 800 for all areas interacting or coming in contact with the face of the person. The body 200 of the protective cover 100 is configured to a predetermined location between the face and the strap 800.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A protective covering for a face of a person utilizing a CPAP/BIPAP therapy device, the covering comprising:
a body, the body having a top and a bottom, wherein the top of the body having a coupling to form the body;
an interior, the interior having an inside edge and an outside edge, wherein the interior having a filling;
a strap coupling, the strap coupling coupled to the body, wherein the strap coupling being removably coupled to a strap of a CPAP/BIPAP mask; and
a first hook and a second hook, the first hook and second hook being coupled to the strap coupling and wherein the first hook being further coupled to the top of the body and wherein the second hook being coupled to a bottom of the body.

2. The covering of claim 1, wherein the body being made of a flannel material.

3. The covering of claim 1, wherein the covering having a length being between four and five (4-5) inches.

4. The covering of claim 1, wherein the protective covering having a height being one (1) inch.

5. The covering of claim 1, wherein the outside edge of the interior having a diameter being one (1) inch.

6. The covering of claim 1, wherein the inside edge of the interior having a diameter being three-fourths (0.75) inch.

7. The covering of claim 1, wherein the filling being polyester.

8. The covering of claim 1, wherein the strap coupling being made of a hook and loop Velcro® material.

9. The covering of claim 1, wherein the coupling being made of a thread material.
